(19) United States
(12) Patent Application Publication
(10) Pub. No.: US 2009/0301521 A1
(43) Pub. Date: Dec. 10, 2009

(54) STEAM CLEANER USING ELECTROLYZED LIQUID AND METHOD THEREFOR

(75) Inventor: Bruce F. Field, Golden Valley, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: Tennant Company, Minneapolis, MN (US)

(21) Appl. No.: 12/481,115

(22) Filed: Jun. 9, 2009

Related U.S. Application Data
(60) Provisional application No. 61/060,407, filed on Jun. 10, 2008.

(51) Int. Cl. B08B 3/10

(52) U.S. Cl. 134/11; 134/107

(57) ABSTRACT

An apparatus and method are provided for generating an electrochemically-activated vapor.

### CROSS-REFERENCE TO RELATED APPLICATION(S)

The present application claims priority to U.S. Provisional Application No. 61/060,407, filed on Jun. 10, 2008, and entitled “STEAM CLEANER USING ELECTROLYZED LIQUID AND METHOD THEREFOR”, the disclosure of which is incorporated by reference in its entirety.

### FIELD OF THE DISCLOSURE

The present disclosure relates to cleaning with electrochemically activated liquids and, more particularly, to an apparatus and method of steam cleaning.

### BACKGROUND

Steam cleaners, referred to as “vapor” or “dry” steam cleaners heat tap water to high temperatures, which transforms the water into a vapor. The vapor is able to enter into small spaces and clean by loosening dirt particles on the object being cleaned. The steam vapor's low moisture content and high temperature provides deeper cleaning because it penetrates into the pores and crevices better than topically applied cleaners. Since steam vapor cleaners use no chemicals, there is a benefit to indoor air quality, no hazards or risks from using and storing chemicals, and no harmful chemical residue left after the cleaning process. Thus, there is an ongoing need to further enhance the cleaning and/or sanitizing properties of steam cleaners.

### SUMMARY

An aspect of the disclosure is directed to a steam cleaning apparatus that includes an electrolysis cell configured to electrochemically activate a liquid, a steam generator configured to at least partially vaporize the electrochemically-activated liquid to produce an electrochemically-activated vapor, and a dispenser configured to dispense the electrochemically-activated vapor.

Another aspect of the disclosure is directed to a method for cleaning. The method includes electrochemically activating a liquid, at least partially vaporizing the electrochemically-activated liquid to produce an electrochemically-activated vapor, and dispensing the electrochemically-activated vapor.

A further aspect of the disclosure is directed to a method for cleaning that includes introducing a first portion of a feed liquid into an anode chamber of an electrolysis cell, introducing a second portion of the feed liquid into a cathode chamber of the electrolysis cell, and applying a voltage potential across the first and second portions of the feed water to electrochemically activate the first and second portions of the feed liquid. The method further includes introducing at least one of the first and second electrochemically-activated portions of the feed liquid to a steam generator, at least partially vaporizing the at least one electrochemically-activated portion to produce an electrochemically-activated vapor, and dispensing the electrochemically-activated vapor.

### BRIEF DESCRIPTION OF THE DRAWINGS

FIG. 1 is a simplified, schematic diagram of a steam generator according to an exemplary aspect of the present disclosure.

FIG. 2 illustrates an example of an electrolysis cell having an ion-selective membrane.

FIG. 3 illustrates an electrolysis cell having no ion-selective membrane according to a further example of the disclosure.

### DETAILED DESCRIPTION

An aspect of the present disclosure is directed to a method and apparatus for steam cleaning using electrolyzed liquid to create the working vapor. Electrolysis cells are used to create anolyte electrochemically activated (EA) liquid and catholyte EA liquid. Anolyte EA liquids have sanitizing properties, and catholyte EA liquids have cleaning properties. Examples of cleaning and/or sanitizing systems are disclosed in Field et al. U.S. Publication No. 2007/0186368 A1, published Aug. 16, 2007. In one aspect of the disclosure, an electrolysis cell is used to produce an electrolyzed liquid, which is then vaporized by a steam cleaner to produce a working steam.

### 1. Steam Cleaner

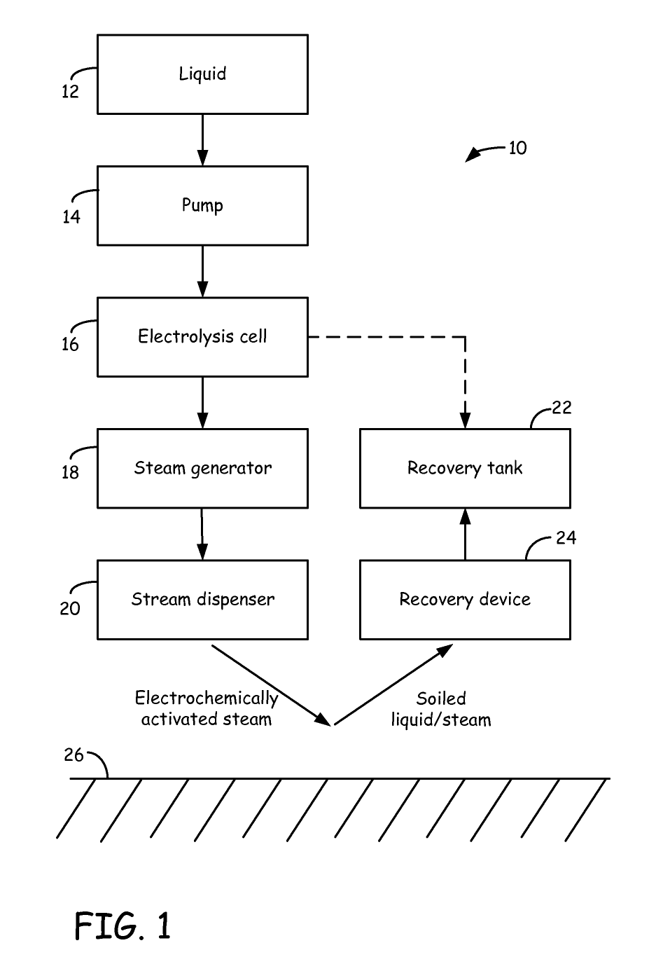

FIG. 1 is a simplified, schematic diagram of a steam cleaner 10 according to an exemplary aspect of the present disclosure. Steam cleaner 10 can include a reservoir 12 or other source, such as a fitting or other liquid input, for containing and/or receiving a working liquid to be treated and then dispensed by the cleaner. In an example, the liquid to be treated includes an aqueous composition, such as regular tap water.

Steam cleaner 10 further includes a pump 14, one or more electrolysis cells 16, a steam generator 18 and a steam dispenser 20 for dispensing electrochemically activated steam. Although not shown in FIG. 1, steam cleaner 10 can also include other elements, such as a power source (e.g., a battery or power cord) one or more control switches, and control electronics for controlling operation of the pump 14, electrolysis cell 16 and steam generator 18.

Pump 14 can be located upstream or downstream of electrolysis cell 16. When energized, pump 14 draws liquid from reservoir 12, through electrolysis cell 18, and into steam generator 18. In one non-limiting example, electrolysis cell 18 converts the liquid to an anolyte EA liquid and a catholyte EA liquid. The anolyte EA liquid and/or the catholyte EA liquid are passed to the steam generator. In one example, one of the anolyte EA liquid or catholyte EA liquid is passed to the steam generator and the other is passed to a recovery or waste tank 22, as shown by the dashed line in FIG. 1. In another example, both the anolyte and catholyte EA liquids are passed to the steam generator as separate streams or as a single, blended stream.

Steam generator 10 can include any suitable type of steam generator, such as a heater immersed in a liquid reservoir. The liquid reservoir contains the electrochemically activated liquid received from electrolysis cell 16. The heater heats water in its immediate vicinity to provide resulting steam to a steam passageway, such as a steam pipe. One end of the steam passageway is connected to the heater and/or the reservoir in the steam generator and the other end is connected to the steam dispenser 20.

In an alternative example, features of the electrolysis cell 16 and the steam generator 18, such as heating elements, reservoir and electrolysis electrodes can be combined in a single device, such that the working liquid becomes electrolyzed and transformed into a vapor within and/or along a combined reservoir, container or flow path. In additional alternative examples, one or both of electrolysis cell 16 and steam generator 18 may include additional reservoirs for retaining liquids for batch operations, or one or both of electrolysis cell 16 and steam generator 18 may be directly fed in a continuous manner.

In one example, the steam produced by steam generator 18 has a low moisture content and is referred to as a “dry steam vapor”. For example, the dry steam vapor has a low percent of liquid by volume, such as but not limited to between about 5% and about 6% liquid by volume. However, the steam can have a water content outside this range in other examples. The vapor is heated to a temperature hot enough, for example, to kill bacteria and germs, emulsify grease and oil as well as other surface contaminates.

Steam dispenser 20 can include any suitable type of dispenser or outlet, such as a jetting nozzle or a simple opening. The dispenser dispenses electrochemically activated steam to a surface or object 26 being cleaned and/or sanitized.

In the example shown in FIG. 1, steam cleaner further includes a recovery device 24, such as a vacuum-type of recovery device, which recovers soiled working liquid and/or steam from the surface being cleaned. In another example, steam cleaner 10 does not include a recovery tank 10 and/or a recovery device 24.

The arrangement shown in FIG. 1 is provided merely as a non-limiting example. Steam cleaner 10 can have any other structural and/or functional arrangement. For example, steam cleaner 10 can be embodied as an upright type of vacuum/steam cleaner, as an attachment to a cleaning device, a cleaning wand, a self-contained cleaning apparatus, etc. For example, with a self-contained cleaning apparatus, liquid source 12 includes a reservoir that is carried by the steam cleaner. In other examples, the liquid reservoir can be external to steam cleaner 10 and connected through a supply tube.

Also, electrolysis cell 16 can be external to steam cleaner 10. In one example, electrolysis cell 16 is implemented as a stand-alone electrolysis cell, which produces anolyte EA liquid, catholyte EA liquid and/or a combined anolyte and catholyte EA liquid. This EA liquid is then inserted into the reservoir of steam cleaner generator 18 by any suitable method.

### 2. Electrolysis Cells

An electrolysis cell includes any fluid treatment cell that is adapted to apply an electric field across the fluid between at least one anode electrode and at least one cathode electrode. An electrolysis cell can have any suitable number of electrodes, any suitable number of chambers for containing the fluid, and any suitable number of fluid inputs and fluid outputs. The cell can be adapted to treat any fluid (such as a liquid or gas-liquid combination). The cell can include one or more ion-selective membranes between the anode and cathode or can be configured without any ion selective membranes. An electrolysis cell having an ion-selective membrane is referred to herein as a “functional generator”.

Electrolysis cells can have a variety of different structures, such as but not limited to the structures disclosed in Field et al. U.S. Patent Publication No. 2007/0186368, published Aug. 16, 2007.

3. Electrolysis Cell having a Membrane

3.1 Cell Structure

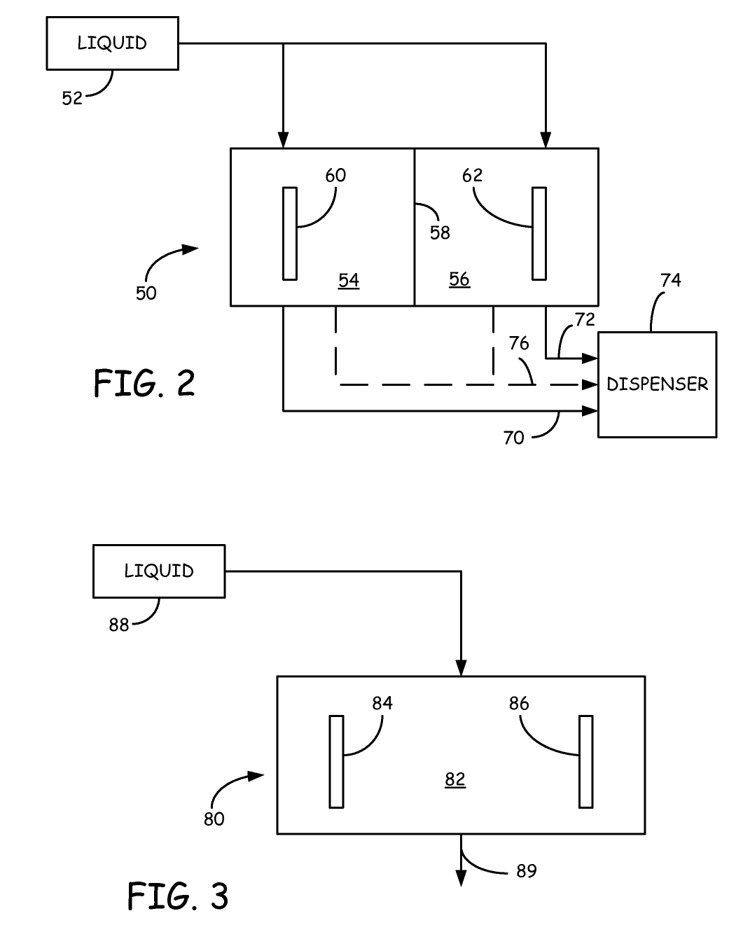

FIG. 2 is a schematic diagram illustrating an example of an electrolysis cell 50 that can be used in or with the steam cleaner shown in FIG. 1, for example. Electrolysis cell 50 and which receives liquid to be treated from a liquid source 52. Liquid source 52 can include a tank or other solution reservoir, such as reservoir 12 in FIG. 1, or can include a fitting or other inlet for receiving a liquid from an external source.

Cell 50 has one or more anode chambers 54 and one or more cathode chambers 56 (known as reaction chambers), which are separated by an ion exchange membrane 58, such as a cation or anion exchange membrane. One or more anode electrodes 60 and cathode electrodes 62 (one of each electrode shown) are disposed in each anode chamber 54 and each cathode chamber 56, respectively. The anode and cathode electrodes 60, 62 can be made from any suitable material, such as titanium and/or titanium coated with a precious metal, such as platinum, or any other suitable electrode material. The electrodes and respective chambers can have any suitable shape and construction. For example, the electrodes can be flat plates, coaxial plates, rods, or a combination thereof. Each electrode can have, for example, a solid construction or can have one or more apertures. In one example, each electrode is formed as a mesh. In addition, multiple cells 50 can be coupled in series or in parallel with one another, for example.

The electrodes 60, 62 are electrically connected to opposite terminals of a conventional power supply (not shown). Ion exchange membrane 58 is located between electrodes 60 and 62. The power supply can provide a constant DC output voltage, a pulsed or otherwise modulated DC output voltage, and/or a pulsed or otherwise modulated AC output voltage to the anode and cathode electrodes. The power supply can have any suitable output voltage level, current level, duty cycle or waveform.

For example in one embodiment, the power supply applies the voltage supplied to the plates at a relative steady state. The power supply includes a DC/DC converter that uses a pulse-width modulation (PWM) control scheme to control voltage and current output. Other types of power supplies can also be used, which can be pulsed or not pulsed and at other voltage and power ranges. The parameters are application-specific. The power supply can be embodied within or external to steam cleaner 10.

During operation, feed water (or other liquid to be treated) is supplied from source 52 to both anode chamber 54 and cathode chamber 56. In the case of a cation exchange membrane, upon application of a DC voltage potential across anode 60 and cathode 62, such as a voltage in a range of about 5 Volts (V) to about 25V, cations originally present in the anode chamber 54 move across the ion-exchange membrane 58 towards cathode 62 while anions in anode chamber 54 move towards anode 60. However, anions present in cathode chamber 56 are not able to pass through the cation-exchange membrane, and therefore remain confined within cathode chamber 56.

While the electrolysis continues, the anions in the liquid bind to the metal atoms (e.g., platinum atoms) at anode 60, and the cations in the liquid bind to the metal atoms (e.g., platinum atoms) at cathode 62. These bound atoms diffuse around in two dimensions on the surfaces of the respective electrodes until they take part in further reactions. Other atoms and polyatomic groups may also bind similarly to the surfaces of anode 60 and cathode 62, and may also subsequently undergo reactions. Molecules such as oxygen (O₂) and hydrogen (H₂) produced at the surfaces may enter small cavities in the liquid phase of the liquid (i.e., bubbles) as gases and/or may become solvated by the liquid phase of the liquid.

Surface tension at a gas-liquid interface is produced by the attraction between the molecules being directed away from the surfaces of anode 60 and cathode 62 as the surface molecules are more attracted to the molecules within the liquid than they are to molecules of the gas at the electrode surfaces. In contrast, molecules of the bulk of the liquid are equally attracted in all directions. Thus, in order to increase the possible interaction energy, surface tension causes the molecules at the electrode surfaces to enter the bulk of the liquid. As a result of the electrolysis process, cell 50 electrochemically activates the feed water by at least partially utilizing electrolysis and produces electrochemically-activated water in the form of an acidic anolyte composition 70 and a basic catholyte composition 72.

If desired, the anolyte and catholyte can be generated in different ratios to one another through modifications to the structure of the electrolysis cell, for example. For example, the cell can be configured to produce a greater volume of catholyte than anolyte if the primary function of the EA water is cleaning. Alternatively, for example, the cell can be configured to produce a greater volume of anolyte than catholyte if the primary function of the EA water is sanitizing. Also, the concentrations of reactive species in each can be varied.

For example, the cell can have a 3:2 ratio of cathode plates to anode plates for producing a greater volume of catholyte than anolyte. Each cathode plate is separated from a respective anode plate by a respective ion exchange membrane. Thus, there are three cathode chambers for two anode chambers. This configuration produces roughly 60% catholyte to 40% anolyte. Other ratios can also be used. The polarities can be reversed to achieve roughly 60% anolyte to 40% catholyte.

3.2 Example Reactions

In addition, water molecules in contact with anode 60 are electrochemically oxidized to oxygen (O₂) and hydrogen ions (H⁺) in the anode chamber 54 while water molecules in contact with the cathode 62 are electrochemically reduced to hydrogen gas (H₂) and hydroxyl ions (OH⁻) in the cathode chamber 56. The hydrogen ions in the anode chamber 54 are allowed to pass through the cation-exchange membrane 58 into the cathode chamber 56 where the hydrogen ions are reduced to hydrogen gas while the oxygen gas in the anode chamber 54 oxygenates the feed water to form the anolyte 70. Furthermore, since regular tap water typically includes sodium chloride and/or other chlorides, the anode 60 oxidizes the chlorides present to form chlorine gas. As a result, a substantial amount of chlorine is produced and the pH of the anolyte composition 70 becomes increasingly acidic over time.

As noted, water molecules in contact with the cathode 62 are electrochemically reduced to hydrogen gas and hydroxyl ions (OH⁻) while cations in the anode chamber 54 pass through the cation-exchange membrane 58 into the cathode chamber 56 when the voltage potential is applied. These cations are available to ionically associate with the hydroxyl ions produced at the cathode 62, while hydrogen gas bubbles form in the liquid. A substantial amount of hydroxyl ions accumulates over time in the cathode chamber 56 and reacts with cations to form basic hydroxides. In addition, the hydroxides remain confined to the cathode chamber 56 since the cation-exchange membrane does not allow the negatively charged hydroxyl ions pass through the cation-exchange membrane. Consequently, a substantial amount of hydroxides is produced in the cathode chamber 56, and the pH of the catholyte composition 72 becomes increasingly alkaline over time.

The electrolysis process in the functional generator 50 allow concentration of reactive species and the formation of metastable ions and radicals in the anode chamber 54 and cathode chamber 56. The electrochemical activation process typically occurs by either electron withdrawal (at anode 60) or electron introduction (at cathode 62), which leads to alteration of physiochemical (including structural, energetic and catalytic) properties of the feed water. It is believed that the feed water (anolyte or catholyte) gets activated in the immediate proximity of the electrode surface where the electric field intensity can reach a very high level. This area can be referred to as an electric double layer (EDL).

### 4. Ion Exchange Membrane

As mentioned above, the ion exchange membrane 58 can include a cation exchange membrane (i.e., a proton exchange membrane) or an anion exchange membrane. Suitable cation exchange membranes for membrane 38 include partially and fully fluorinated ionomers, polyaromatic ionomers, and combinations thereof. Examples of suitable commercially available ionomers for membrane 38 include sulfonated tetrafluorethylene copolymers available under the trademark “NAFION” from E.I. du Pont de Nemours and Company, Wilmington, Del.; perfluorinated carboxylic acid ionomers available under the trademark “FLEMION” from Asahi Glass Co., Ltd., Japan; perfluorinated sulfonic acid ionomers available under the trademark “ACIPLEX” Aciplex from Asahi Chemical Industries Co. Ltd., Japan; and combinations thereof. However, any ion exchange membrane can be used in other examples.

### 5. Dispenser

The anolyte and catholyte EA liquid outputs can be coupled to a dispenser 74, which can include any type of dispenser or dispensers, such as an outlet, fitting, spigot, spray head, a cleaning/sanitizing tool or head, etc. In the example shown in FIG. 1, dispenser 34 includes spray nozzle 20, which dispenses the electrochemically activated liquid after being transformed into a vapor by steam generator 18 (not shown in FIG. 2). There can be a dispenser for each output 70 and 72 or a combined dispenser for both outputs.

In one example, the anolyte and catholyte outputs are blended into a common output stream 76, which is supplied to steam generator 20 and then dispenser 74. As described in Field et al. U.S. Patent Publication No. 2007/0186368, it has been found that the anolyte and catholyte can be blended together within the distribution system of a cleaning apparatus and/or on the surface or item being cleaned while at least temporarily retaining beneficial cleaning and/or sanitizing properties. Although the anolyte and catholyte are blended, they are initially not in equilibrium and therefore temporarily retain their enhanced cleaning and sanitizing properties.

It is also believed that the anolyte and catholyte EA liquids retain enhanced cleaning and/or sanitizing properties after being heated and vaporized by a steam generator. Thus, the produced steam has an increased cleaning/sanitizing efficiency.

6. Electrolysis Cell with no Ion-Selective Membrane

FIG. 3 illustrates an electrolysis cell 80 having no ion-selective membrane according to a further example of the disclosure. Cell 80 includes a reaction chamber 82, an anode 84 and a cathode 86. Chamber 82 can be defined by the walls of cell 80, by the walls of a container or conduit in which electrodes 84 and 86 are placed, or by the electrodes themselves, for example. Anode 84 and cathode 86 may be made from any suitable material or a combination of materials, such as titanium and/or titanium coated with a precious metal, such as platinum. Anode 84 and cathode 86 are connected to a conventional electrical power supply. In one embodiment, electrolytic cell 80 includes its own container that defines chamber 82 and is located in the flow path of the liquid to be treated, such as within the flow path of a steam cleaner.

During operation, liquid is supplied by a source 88 and introduced into reaction chamber 82 of electrolysis cell 80. In the embodiment shown in FIG. 3, electrolysis cell 80 does not include an ion exchange membrane that separates reaction products at anode 84 from reaction products at cathode 86. In the example in which tap water is used as the liquid to be treated for use in cleaning, after introducing the water into chamber 82 and applying a voltage potential between anode 84 and cathode 86, water molecules in contact with or near anode 84 are electrochemically oxidized to oxygen (O₂) and hydrogen ions (H⁺) while water molecules in contact or near cathode 86 are electrochemically reduced to hydrogen gas (H₂) and hydroxyl ions (OH⁻). Other reactions can also occur and the particular reactions depend on the components of the liquid. The reaction products from both electrodes are able to mix and form an oxygenated fluid 89 (for example) since there is no physical barrier, for example, separating the reaction products from each other. Alternatively, for example, anode 84 can be separated from cathode 84 by using a dielectric barrier such as a non-permeable membrane (not shown) disposed between the anode and cathode.

### 7. Control Circuit

Referring back to FIG. 1, steam cleaner 10 can include any suitable control circuit, which can be implemented in hardware, software, or a combination of both, for example. The control circuit can be configured to power and control the operation of pump 14, electrolysis cell 16, and/or steam generator 18. In one example, the control circuit includes a power supply having an output that is coupled to pump 14, electrolysis cell 16 and steam generator 18 and which controls the power delivered to the devices.

In one example the control circuit activates pump 14, electrolysis cell 16 and steam generator 18 in response to actuation of a user switch so that steam generator 10 produces electrochemically activated steam in an “on demand” fashion. When the switch is not actuated, pump 14 is in an “off” state and electrolysis cell 16 and steam generator 18 are de-energized. When the switch is actuated to a closed state, the control circuit switches pump 14 to an “on” state and energizes electrolysis cell 16 and steam generator 18. In the “on” state, pump 14 pumps water from reservoir 12 through cell 14 and into steam generator 18.

Other activation sequences can also be used. For example, the control circuit can be configured to energize electrolysis cell 16 for a period of time before energizing steam generator 18 in order to allow the reservoir in steam generator 18 to begin to fill with electrochemically activated water before energizing the heating element.

The control circuit can also include an H-bridge, for example, that is capable of selectively reversing the polarity of the voltage applied to electrolysis cell 16 as a function of a control signal generated by the control circuit. For example, the control circuit can be configured to alternate polarity in a predetermined pattern, such as every 5 seconds with a 50% duty cycle. Other frequencies and duty cycles can be used in alternative embodiments. Frequent reversals of polarity can provide a self-cleaning function to the electrodes, which can reduce scaling or build-up of deposits on the electrode surfaces and can extend the life of the electrodes.

The electrodes of the electrolysis cell can be driven with a variety of different voltage and current patterns, depending on the particular application of the cell. It is desirable to limit scaling on the electrodes by periodically reversing the voltage polarity that is applied to the electrodes. Therefore, the terms “anode” and “cathode” and the terms “anolyte” and “catholyte” as used in the description and claims are respectively interchangeable. This tends to repel oppositely-charged scaling deposits.

In one example, the electrodes are driven at one polarity for a specified period of time (e.g., about 5 seconds) and then driven at the reverse polarity for approximately the same period of time. If the anolyte and catholyte EA liquids are blended at the outlet of the cell, this process produces essentially one part anolyte EA liquid to one part catholyte EA liquid.

If the outputs are not blended, valving can be used, if desired, to maintain a substantially constant anolyte EA liquid at one outlet and a substantially constant catholyte EA liquid at another outlet, wherein the valving switches states with each switch in polarity so that the anolyte and catholyte liquids are always routed to the same outlet even though the electrolysis chambers switch from anode-to-cathode and vice versa.

If the number of anode electrodes is different than the number of cathode electrodes, e.g., a ratio of 3:2, then the electrolysis cell can be used to produce a greater amount of either anolyte or catholyte, if desired, to emphasize cleaning or sanitizing properties of the produced liquid. For example, if cleaning is to be emphasized, then a greater number of electrodes can be driven to a relatively negative polarity (to produce more catholyte) and a lesser number of electrodes can be driven to the relatively positive polarity (to produce less anolyte). If sanitizing is to be emphasized, then a greater number of electrodes can be driven to the relatively positive polarity (to produce more anolyte) and a lesser number of electrodes can be driven to the relatively negative polarity (to produce less catholyte).

If the anolyte and catholyte outputs are blended into a single output stream prior to dispensing, then the combined anolyte and catholyte output liquid can be tailored to emphasize cleaning over sanitizing or to emphasize sanitizing over cleaning.

Although the present disclosure has been described with reference to one or more embodiments, workers skilled in the art will recognize that changes may be made in form and detail without departing from the spirit and scope of the disclosure and/or the appended claims.

1. A steam cleaning apparatus comprising:
an electrolysis cell configured to electrochemically activate a liquid;
a steam generator configured to at least partially vaporize the electrochemically-activated liquid to produce an electrochemically-activated vapor; and
a dispenser configured to dispense the electrochemically-activated vapor.

2. The steam cleaning apparatus of claim 1, wherein the electrolysis cell comprises:
a chamber;
an anode electrode disposed within the chamber, and configured to be electrically connected to a power source; and
a cathode electrode disposed within the chamber, and configured to be electrically connected to the power source.

3. The steam cleaning apparatus of claim 2, wherein the electrolysis cell further comprises an ion exchange membrane disposed between the anode electrode and the cathode electrode.

4. The steam cleaning apparatus of claim 1, and further comprising a recovery device configured to recover soiled working liquid from a surface being cleaned.

5. The steam cleaning apparatus of claim 1, wherein the steam generator comprises:
a liquid reservoir configured to receive the electrochemically-activated liquid from the electrolysis cell; and
a heater immersed in the liquid reservoir and configured to heat the received electrochemically-activated liquid, thereby at least partially vaporizing the electrochemically-activated liquid.

6. The steam cleaning apparatus of claim 1, and further comprising a recovery tank, wherein at least a portion of the electrochemically-activated liquid is directed to the recovery tank.

7. A method for cleaning, the method comprising:
electrochemically activating a liquid;
at least partially vaporizing the electrochemically-activated liquid to produce an electrochemically-activated vapor; and
dispensing the electrochemically-activated vapor.

8. The method of claim 7, wherein the liquid is electrochemically activated in at least one electrolysis cell.

9. The method of claim 7, wherein electrochemically activating the liquid comprises:
introducing a feed liquid into an electrolysis cell, the electrolysis cell having at least one cathode electrode and at least one anode electrode; and
applying a voltage potential across the at least one cathode electrode and the at least one anode electrode to generate the electrochemically-activated liquid from the feed liquid.

10. The method of claim 9, and further comprising maintaining separation of at least two portions of the feed liquid with at least one ion exchange membrane disposed between the at least one cathode electrode and the at least one anode electrode.

11. The method of claim 7, wherein the liquid consists essentially of water.

12. The method of claim 7, wherein the electrochemically-activated liquid is selected from the group consisting of an anolyte electrochemically-activated liquid, a catholyte electrochemically-activated liquid, and a combination thereof.

13. The method of claim 7, wherein the electrochemically-activated vapor is a dry steam vapor.

14. A method for cleaning, the method comprising:
introducing a first portion of a feed liquid into an anode chamber of an electrolysis cell;
introducing a second portion of the feed liquid into a cathode chamber of the electrolysis cell;
applying a voltage potential across the first and second portions of the feed water to electrochemically activate the first and second portions of the feed liquid;
introducing at least one of the first and second electrochemically-activated portions of the feed liquid to a steam generator;
at least partially vaporizing the at least one electrochemically-activated portion to produce an electrochemically-activated vapor; and
dispensing the electrochemically-activated vapor.

15. The method of claim 14, and further comprising maintaining separation of the anode chamber and the cathode chamber within the electrolysis cell with an ion exchange membrane.

16. The method of claim 14, wherein the feed liquid consists essentially of water.

18. The method of claim 15, and further comprising blending the first and second electrochemically-activated portions of the feed liquid prior to being fed to the steam generator.

19. The method of claim 15, wherein the other of the at least one of the first and second electrochemically-activated portions of the feed liquid is directed to a recovery tank.

20. The method of claim 15, wherein at least partially vaporizing the at least one electrochemically-activated portion comprises:
receiving the at least one electrochemically-activated portion in a liquid reservoir; and
heating the at least one electrochemically-activated portion in a liquid reservoir with a heater immersed in the liquid reservoir.
